(19) United States
(12) Patent Application Publication
(10) Pub. No.: US 2022/0117720 A1
(43) Pub. Date: Apr. 21, 2022

(54) LIGAMENT REPAIR SYSTEM AND METHOD

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Jennifer Ng, Seattle, WA (US); Siraaj Dhru, Monmouth Beach, NJ (US); Robert F. LaPrade, Avon, CO (US)

(73) Assignee: Smith & Nephew, Inc., Memphis, TN (US)

(21) Appl. No.: 17/505,765

(22) Filed: Oct. 20, 2021

Related U.S. Application Data
(60) Provisional application No. 63/093,932, filed on Oct. 20, 2020.

(51) Int. Cl. A61F 2/08; A61B 17/17

(52) U.S. Cl. CPC A61F 2/0805; A61B 17/1764; A61B 2017/00969; A61F 2002/0888; A61F 2/0811

(57) ABSTRACT

Embodiments of the disclosure are directed to a ligament repair system and method. The ligament repair system may include one or more of a femoral aimer assembly, a tibial aimer assembly, a femoral fixation assembly, and a tibial fixation assembly.

### CROSS-REFERENCE TO RELATED APPLICATIONS

This application claims priority to and benefit of co-pending U.S. Provisional Application No. 63/093,932, filed Oct. 20, 2020, entitled LIGAMENT REPAIR SYSTEM AND METHOD, the entire contents of which are incorporated herein by reference for all purposes.

### FIELD

The present disclosure relates generally to the field of medical devices instruments, and more particularly relates systems and methods used to repair or replace ligaments. Example systems and methods may be used to repair or replace a posterior cruciate ligament (PCL), including separately coupling separate bundles of the PCL.

### BACKGROUND

When a ligament, such as a PCL, undergoes trauma or other injury or conditions, the ligament may become permanently lax or may be torn. In such a circumstance, the ligament is unable to function properly. In the case of a PCL dysfunction, the tibia may translate posteriorly relative to the femur, and the stability of the knee may be greatly decreased. In order to restore biomechanical function, the PCL, or a substituted graft, must be securely fixed within the knee to an anatomically correct position. Anatomically correct positioning may be achieved by locating a graft at native tibial and femoral footprints.

What is needed are instruments, implants, and methods configured to provide for accurate placement and fixation of a ligament graft, or multiple bundles of a ligament graft, at anatomically correct positions. An improved system may accomplish this without creating one or more posterior portals, which have higher associated surgical risk. An improved system may also allow for tensioning to be performed tibially instead of femorally, which allows a surgeon to spend minimal time operating inside of the knee joint space. An improved system may also provide for tibial tensioning of more than one graft bundle to be accomplished in a single tunnel, but with separate tensions being applied to each graft bundle. An improved system may also reduce the risk of potential damage to grafts by providing impacted or “pound-in” fixation devices for some embodiments. Such improved systems may also avoid requiring a graft to be wrapped around a screw or requiring the use of spiked washers, which may damage a graft by piercing the graft.

### SUMMARY

An embodiment of the disclosure is a ligament repair system that includes a femoral aimer assembly, a tibial aimer assembly, a femoral fixation assembly, and a tibial fixation assembly. The femoral aimer assembly may include a handle and a cannula configured to couple with the handle. The cannula may include a cannulation with a longitudinal axis through which a guidewire may be inserted into a femur. Femoral aimer embodiments may also include a protrusion at a distal end of the cannula with a center that is offset from a center of the longitudinal axis of the cannulation, where the protrusion may be placed in a hole in the femur. The offset distance between the protrusion and the longitudinal axis of the cannulation of some embodiments defines a predetermined distance between the hole in the femur and a guidewire that may be inserted through the cannulation. The tibial aimer assembly may include a base, a drill guide coupled to the base, a left aimer arm configured to releasably couple with the base, and a right aimer arm configured to releasably couple with the base. The left aimer arm may more particularly include a first leg configured to releasably couple with the base in a common plane with the base, and a left extension with a first main axis that extends from its proximal end to its distal tip, wherein the proximal end of the left extension is fixed to the first leg, a central portion of the left extension diverts to the left of the first main axis, and the distal tip of the left extension is configured to align with a central axis of the drill guide. The right aimer arm may more particularly include a second leg configured to releasably couple with the base in a common plane with the base, and a right extension with a second main axis that extends from its proximal end to its distal tip, wherein the proximal end of the right extension is fixed to the second leg, a central portion of the right extension diverts to the right of the second main axis, and the distal tip of the right extension is configured to align with a central axis of the drill guide. The femoral fixation assembly may include a first fixation device for coupling a first ligament bundle to the femur, and a second fixation device for coupling a second ligament bundle to the femur. The tibial fixation assembly may include an exterior anchor with a proximal end, a distal end, and an interior opening. The exterior anchor may be configured to couple the first ligament bundle between an exterior portion of the exterior anchor and a hole in the tibia. The internal anchor may fit substantially within the interior opening in the exterior anchor to couple the second ligament bundle relative to the exterior anchor.

Another embodiment of the disclosure is a femoral aimer assembly that includes a handle and a cannula configured to couple with the handle. The cannula has a cannulation with a longitudinal axis through which a guidewire may be inserted into a femur. The femoral aimer assembly may also include a protrusion at a distal end of the cannula with a center that is offset from a center of the longitudinal axis of the cannulation. When the protrusion is placed in a hole in the femur, the offset distance between the protrusion and the longitudinal axis of the cannulation defines a predetermined distance between the hole and a guidewire that may be inserted through the cannulation.

Yet another embodiment of the disclosure is a tibial aimer assembly that includes a base, a drill guide coupled to the base, a left aimer arm configured to releasably couple with the base, and a right aimer arm configured to releasably couple with the base. The left aimer arm may include a first leg configured to releasably couple with the base in a common plane with the base, and a left extension with a first main axis that extends from its proximal end to its distal tip. In some embodiments, the proximal end of the left extension is fixed to the first leg, a central portion of the left extension diverts to the left of the first main axis, and the distal tip of the left extension is configured to align with a central axis of the drill guide. The right aimer arm may include a second leg configured to releasably couple with the base in a common plane with the base, and a right extension with a second main axis that extends from its proximal end to its distal tip. In some embodiments, the proximal end of the right extension is fixed to the second leg, a central portion of the right extension diverts to the right of the second main axis, and the distal tip of the right extension is configured to align with a central axis of the drill guide.

Another embodiment of the disclosure is a tibial fixation assembly that includes an exterior anchor with a proximal end, a distal end, and an interior opening. The exterior anchor may be configured to couple a first ligament bundle between an exterior portion of the exterior anchor and a hole in the tibia. The tibial fixation assembly may also include an internal anchor that fits substantially within the interior opening in the exterior anchor to couple a second ligament bundle relative to the exterior anchor.

Still another embodiment of the disclosure is a method of implanting a posterior cruciate ligament (PCL) replacement graft in a patient's knee. The method may include fixing an anterolateral bundle (ALB) graft and a posteromedial bundle (PMB) graft to the patient's femur and tibia. Tibial fixing may include preparing the patient's tibia to receive the ALB graft and the PMB graft and then positioning the ALB graft and the PMB graft through the tibia for fixation. The patient's knee may then be positioned at about 90 degrees of flexion, and the ALB may be tensioned relative to the tibia. The ALB may then be fixed relative to the tibia by placing an exterior anchor with a proximal end, a distal end, and an interior opening in a tibial tunnel prepared in the patient's tibia in which the ALB is located. The method may also include positioning the patient's knee at about zero degrees of flexion, tensioning the PMB relative to the tibia, and fixing the PMB relative to the tibia. Fixation may be accomplished by placing an interior anchor in the interior opening of the exterior anchor in which the PMB is located.

A reading of the following detailed description and a review of the associated drawings will make apparent the advantages of these and other features. Both the foregoing general description and the following detailed description serve as an explanation only and do not restrict aspects of the disclosure as claimed.

### BRIEF DESCRIPTION OF THE DRAWINGS

Reference to the detailed description, combined with the following figures, will make the disclosure more fully understood, wherein:

FIG. 1 is a side elevation view of an embodiment of a femoral aimer assembly in a first configuration.

FIG. 2 is a side elevation view of an embodiment of a femoral aimer assembly in a second configuration.

FIG. 3 is a perspective view of a handle of the femoral aimer assembly of FIGS. 1 and 2.

FIG. 4 is a cross-sectional view of the handle shown in FIG. 3.

FIG. 5 is a perspective view of a cannula of the femoral aimer assembly of FIGS. 1 and 2.

FIG. 6 is a perspective view of an embodiment of the femoral aimer assembly in use on a femur.

FIG. 7 is a perspective view of an embodiment of the femoral aimer assembly in further use on a femur.

FIG. 8 is a plan view of an embodiment of a tibial aimer assembly with a left aimer arm in use on a tibia.

FIG. 9 is a perspective view of the tibial aimer assembly shown in FIG. 8.

FIG. 10 is a perspective view of an embodiment of a tibial aimer assembly with a right aimer arm and with some components removed for clarity.

FIG. 11 is a plan view of the tibial aimer assembly with a left aimer arm of FIG. 8 with some components removed for clarity.

FIG. 12 is a plan view of the tibial aimer assembly with a right aimer arm of FIG. 10 with some components removed for clarity.

FIG. 13 is a perspective view of some components of an embodiment of a tibial fixation assembly in combination with instruments for inserting the tibial fixation assembly.

FIG. 14 is a perspective view of an insertion cannula instrument for inserting components of the tibial fixation assembly illustrated in FIG. 13 in position on the tibia.

FIG. 15 is a perspective view of an inserter instrument and an exterior anchor of the tibial fixation assembly illustrated in FIG. 13.

FIG. 16 is a distal quarter perspective view of an embodiment of an exterior anchor of the tibial fixation assembly illustrated in FIG. 13.

FIG. 17 is a proximal quarter perspective view of an embodiment of an exterior anchor of the tibial fixation assembly illustrated in FIG. 13.

FIG. 18 is a perspective view of another embodiment of an inserter instrument and another embodiment of an exterior anchor of a tibial fixation assembly.

FIG. 19 is an enlarged perspective view of a distal end of the inserter instrument and exterior anchor illustrated in FIG. 18.

FIG. 20 is a perspective view of the distal end of the inserter instrument illustrated in FIG. 19.

FIG. 21 is a distal quarter perspective view of the exterior anchor illustrated in FIG. 18.

FIG. 22 is a proximal quarter perspective view of the exterior anchor illustrated in FIG. 18.

FIG. 23 is a perspective view of the exterior anchor illustrated in FIG. 18 implanted in a tibial tunnel formed in the tibia and with graft material exiting the tibial tunnel.

FIG. 24 is an elevation view of the exterior anchor illustrated in FIG. 18 and an interior anchor implanted in a tibial tunnel formed in a tibia and with graft material exiting the tibial tunnel.

### DETAILED DESCRIPTION

In the following description, like components have the same reference numerals, regardless of different illustrated examples. To illustrate examples clearly and concisely, the drawings may not necessarily reflect appropriate scale and may have certain features shown in somewhat schematic form. The disclosure may describe and/or illustrate features in one example, and in the same way or in a similar way in one or more other examples, and/or combined with or instead of the features of the other examples.

In the specification and claims, for the purposes of describing and defining the disclosure, the terms “about” and “substantially” represent the inherent degree of uncertainty attributed to any quantitative comparison, value, measurement, or other representation. The terms “about” and “substantially” moreover represent the degree by which a quantitative representation may vary from a stated reference without resulting in a change in the basic function of the subject matter at issue. Open-ended terms, such as “comprise,” “include,” and/or plural forms of each, include the listed parts and can include additional parts not listed, while terms such as “and/or” include one or more of the listed parts and combinations of the listed parts.

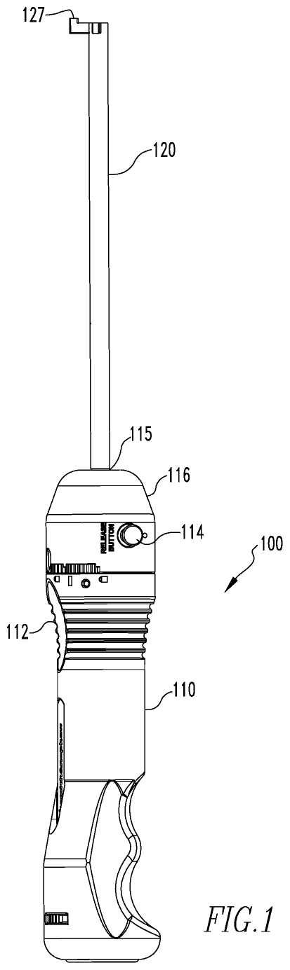

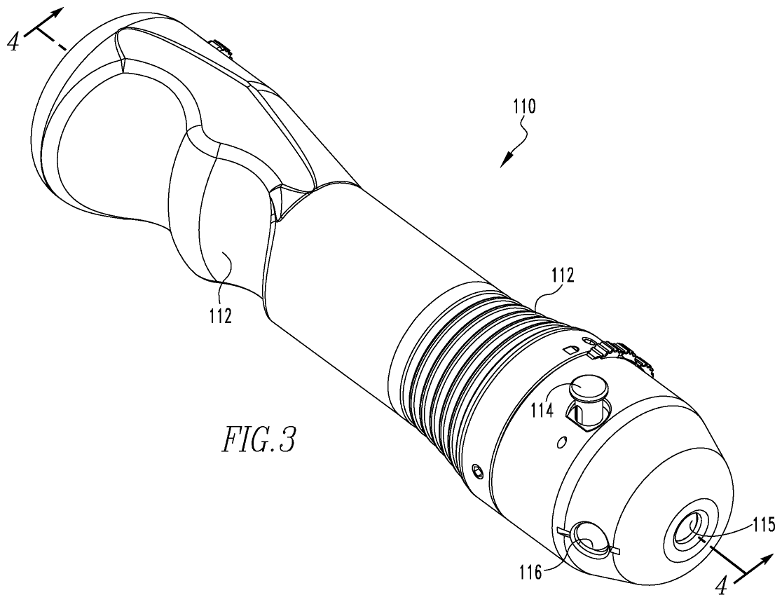

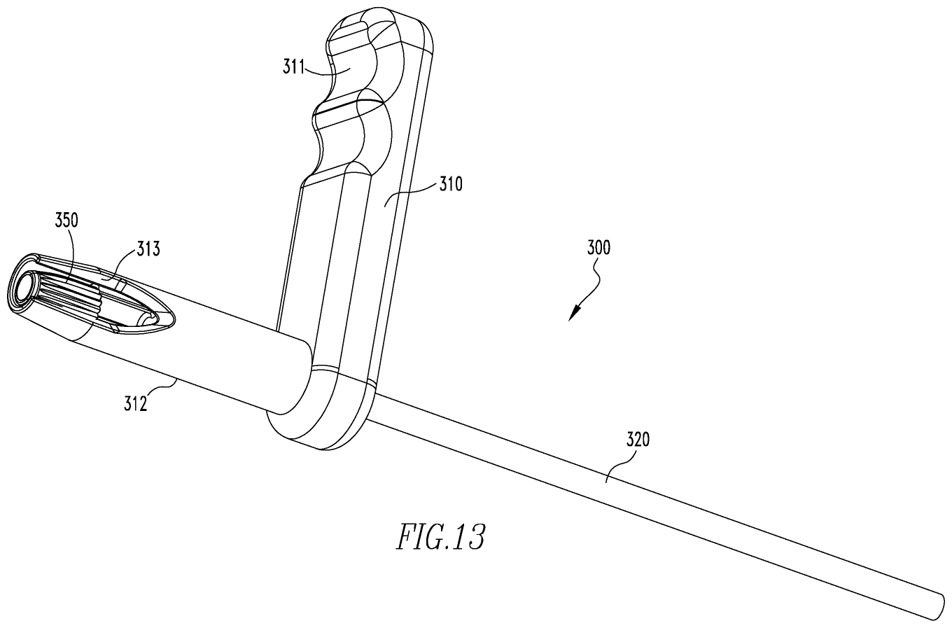

An embodiment of the disclosure is a ligament repair system that includes collectively, a femoral aimer assembly 100, as shown in FIGS. 1-7, a tibial aimer assembly 200, as shown in FIGS. 8-12, a femoral fixation assembly that includes first and second fixation devices, and a tibial fixation assembly 300, as shown in FIGS. 13-24. The components of the system may be used to perform a method of implanting a posterior cruciate ligament (PCL) replacement graft, as will be further described herein.

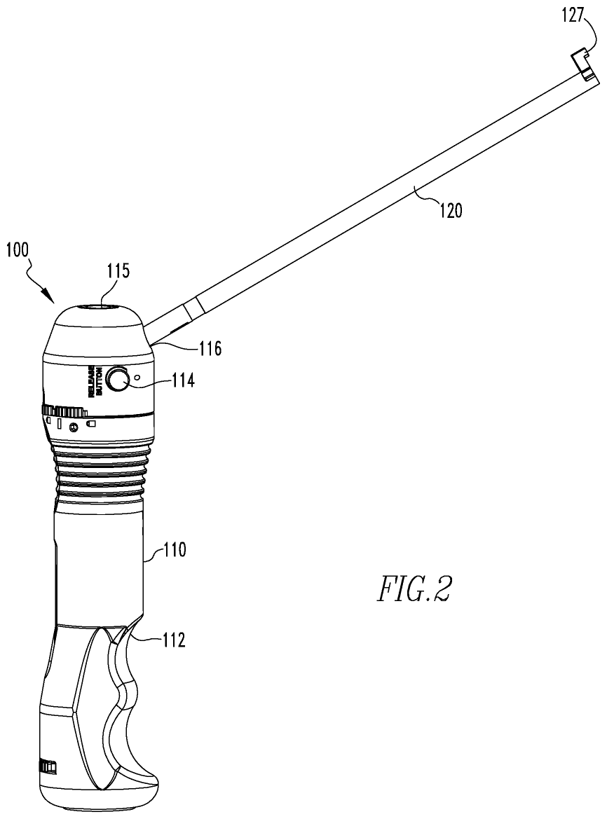

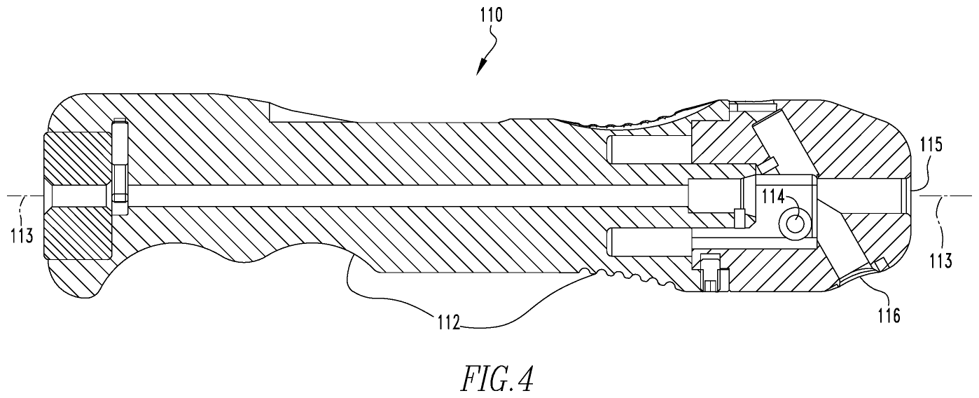

An embodiment of the femoral aimer assembly 100 is illustrated in FIGS. 1-7. A handle 110 is depicted in FIGS. 1-4 and includes at least a gripping surface 112 and a release button 114. The release button 114 is configured to release a cannula 120 from either a first cannula opening 115 or a second cannula opening 116 when pressed. The example release button 114 is spring biased to create a locked connection between the handle 110 and the cannula 120 when the release button 114 is not pressed and the cannula 120 is in either the first cannula opening 115 or the second cannula opening 116. The cannula 120 is shown in the first cannula opening 115 in FIG. 1 and in the second cannula opening 116 in FIG. 2. In the first cannula opening 115, a longitudinal axis of the cannula 120 and the cannulation 122 are coupled to the handle 110 at an angle of about zero degrees with a longitudinal axis 113 of the handle 110 (FIGS. 1 and 4). In the second cannula opening 116, the cannula 120 and the cannulation 122 are coupled to the handle 110 at an angle of about 45 degrees to a longitudinal axis 113 of the handle 110 (FIGS. 2 and 4). The two angle choices provided by this configuration afford a user of the instrument greater flexibility in how an operative site may be approached.

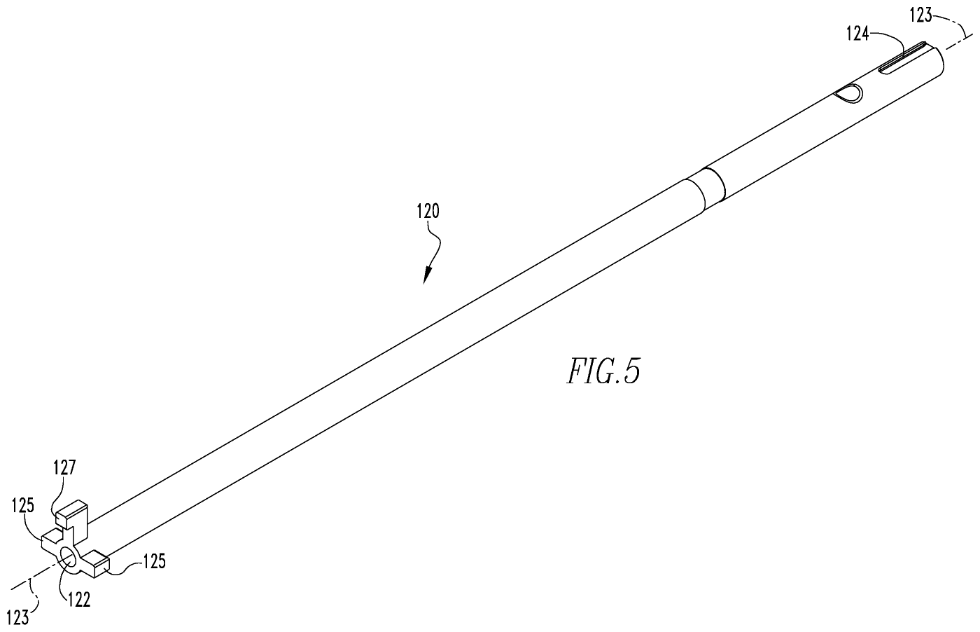

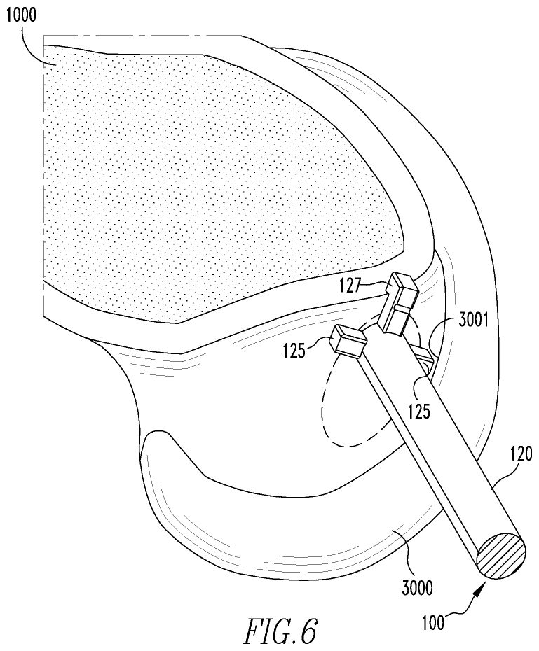

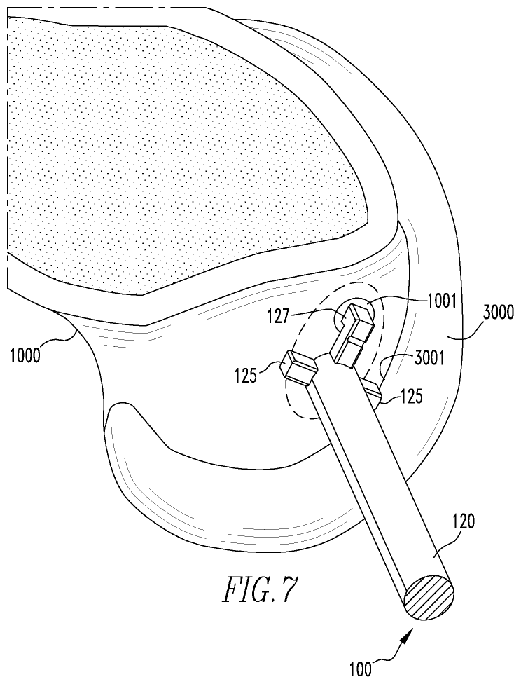

The cannula 120 shown in FIG. 5 includes a cannulation 122 with a longitudinal axis 123 through which a guidewire may be inserted, for example, into a femur. The cannula 120 of the example embodiment includes a guide slot 124 useful in rotationally aligning the cannula 120 with the handle about the longitudinal axis 123 relative to the handle 110, whether the cannula is being placed in the first cannula opening 115 or the second cannula opening 116. The cannula 120 illustrated in FIGS. 1, 2, and 5-7 also includes a protrusion 127 at a distal end of the cannula 120 with a center that is offset from a center of the longitudinal axis 123 of the cannulation 122. Two spurs 125 are also shown at the distal end of the cannula 120 with respective distal ends that are a given distance from the center of the longitudinal axis 123. In this example, the given distance is 5 mm, as is written on the spurs 125 in FIGS. 6 and 7. In other embodiments, the distance may be between about 2 mm and 8 mm. The spurs 125 may be used to consistently establish the center of the longitudinal axis 123 of the cannulation the given distance (5 mm in this example) from an edge 3001 of a cartilage to bone interface 3000, as shown in FIGS. 6 and 7. The spurs 125 extend in opposite directions so that the same femoral aimer assembly 100 may be used in either a right knee or a left knee surgery.

In some embodiments, the protrusion 127 may be substantially the same cross-sectional size or diameter as the guidewire inserted through the cannulation 122 and into the femur. In other embodiments, the protrusion 127 may be substantially the same cross-sectional size or diameter as a reamed hole in the femur 1000. For example, a hole 1001 (FIG. 7) or reamed hole may be between about 2 mm and about 11 mm. A reamer used to create a reamed hole in the femur may have a diameter, for example, of between about 7 mm and about 11 mm. The reamer may be cannulated to fit over a guidewire previously placed in the femur 1000. When the protrusion 127 is placed in the hole 1001 in the femur 1000, as illustrated in FIG. 7, the offset distance between the center of the protrusion 127 and the longitudinal axis 123 of the cannulation 122 defines a predetermined distance between the hole 1001 and a guidewire (not shown). The guidewire may be within the cannulation 122 of the cannula 120 where the cannula 120 is shown positioned in FIG. 7. By this method, two substantially evenly spaced, or even substantially parallel holes, may be consistently established in the femur 1000. In some embodiments, the femoral aimer assembly 100 is configured and sized such that when a first hole 1001 has been made in the femur 1000 to accept the protrusion 127 and a second hole (not shown, but as positioned in FIG. 7) has been made in the femur 1000 with the protrusion 127 in the first hole 1001, and the first and second holes have been reamed, a bone bridge of about at least 2 mm exists between the first hole 1001, or the opening around the first hole 1001 after reaming, and the second hole after reaming.

The tibial aimer assembly 200 illustrated in FIGS. 8-12 includes a base 201, a drill guide 210 coupled to the base 201, a left aimer arm 230 (FIGS. 8, 9, and 11) configured to releasably couple with the base 201, and a right aimer arm 240 (FIGS. 10 and 12) configured to releasably couple with the base 201. Embodiments of a tibial aimer assembly may include only a left aimer arm 230 or only a right aimer arm 240 or may include both as part of the assembly. The drill guide 210 illustrated includes threads 212 that engage with the base 201 to enable the drill guide 210 to be moved relative to the base 201 by turning the drill guide 210 about its longitudinal axis. Other embodiments of a drill guide may include other effective movement and fixing mechanisms. The drill guide 210 depicted also includes a sharpened distal end 214 that is configured to cut into and secure against bone when the drill guide 210 is advanced against the bone. The drill guide 210 in the illustrated embodiment includes a center opening through which a guidewire 260 may be advance to drill through a bone such as a tibia 2000, as illustrated in FIG. 8. The guidewire 260 may be, by way of non-limiting example, a 2.4 mm k-wire.

The left aimer arm 230 shown includes a first leg 231 configured to releasably couple with the base 201 in a common plane with the base 201. In the illustrated embodiment, a knob 211 is located on the base 201 such that when the knob 211 is tightened, sliding movement between the first leg 231 of the left aimer arm 230 along the arc of the first leg 231 is restricted. When the knob 211 is loosened, the first leg 231 is released and allowed to slide relative to the base 201 along the arc of the first leg 231. The first leg 231 of the left aimer arm 230 of the illustrated embodiment is shaped in cooperation with the base 201 such that when the first leg 231 of the left aimer arm 230 is moved along its arc, the distal tip 237 of a left extension 235 remains aligned with an extended central axis of the drill guide 210.

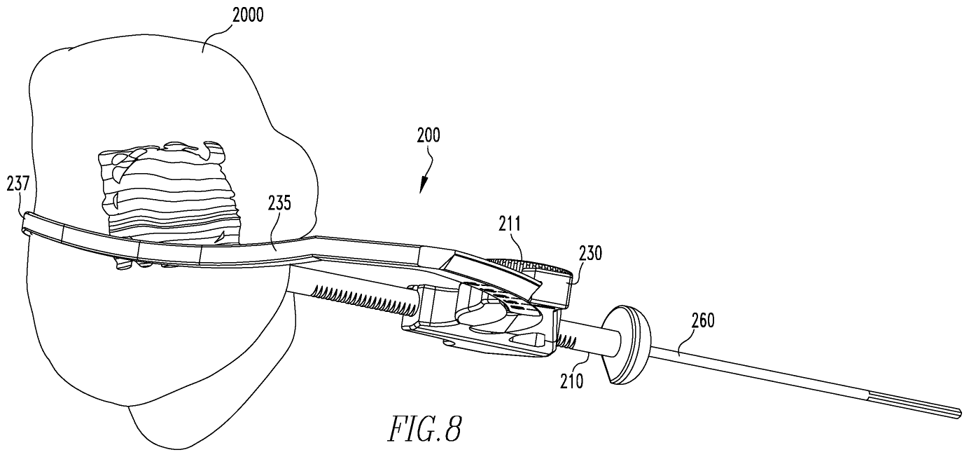

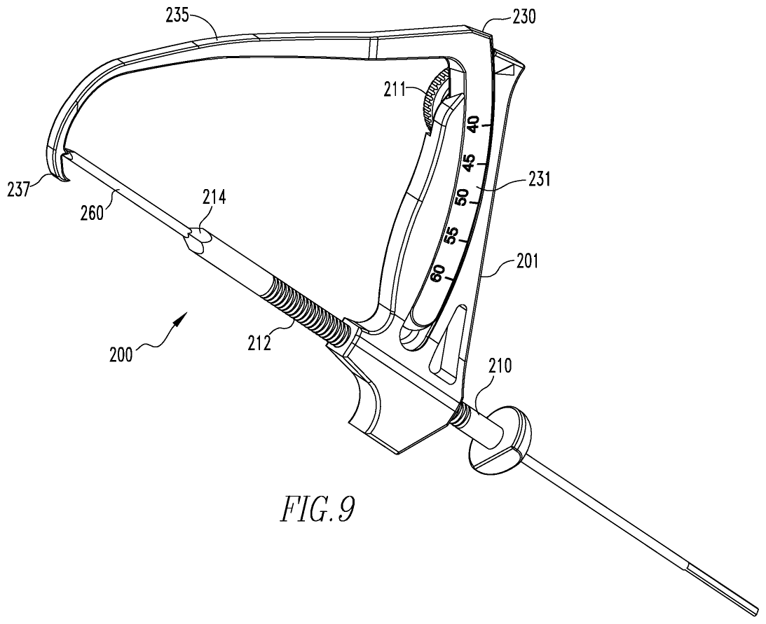

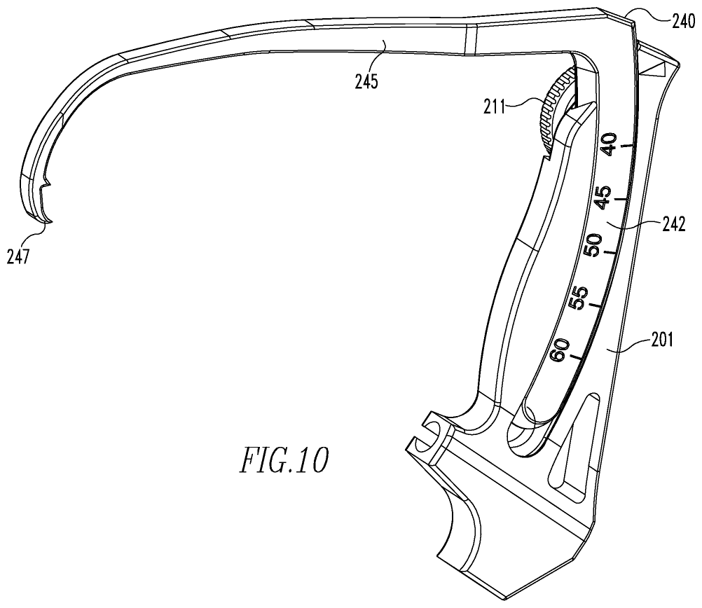

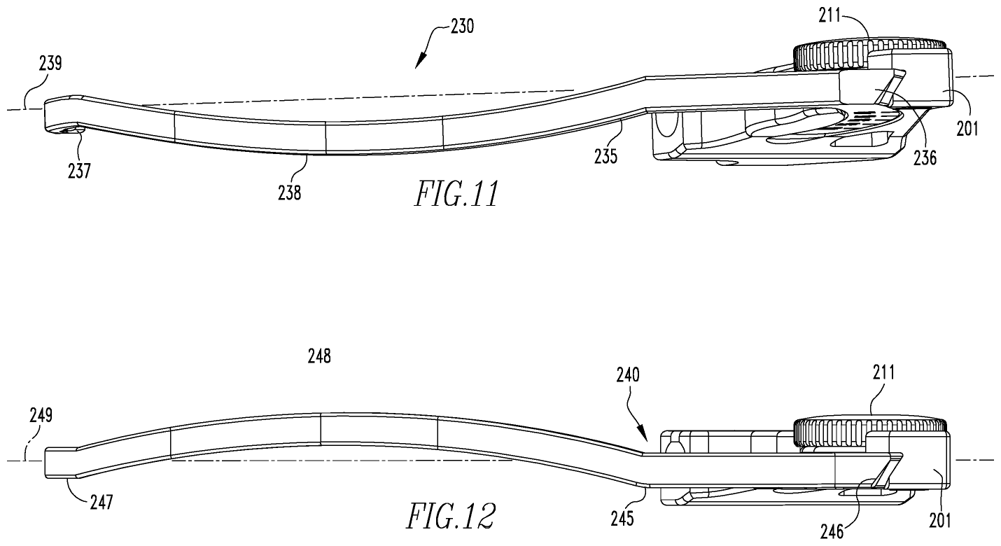

The left aimer arm 230 shown includes the left extension 235 with a first main axis 239 (FIG. 11) that extends from its proximal end 236 to its distal tip 237. In the illustrated embodiment, the proximal end 236 of the left extension 235 is fixed to the first leg 231, a central portion 238 of the left extension 235 diverts to the left of the first main axis 239, and the distal tip 237 of the left extension 235 is configured to align with a central axis of the drill guide 210. The central axis of the drill guide 210, as projected along the guidewire 260 (FIG. 9), is shown aligned with the distal tip 237 in this embodiment. The distal tip 237 is configured to receive or “catch” a distal portion of the guidewire 260, which is useful in preventing penetration of the guidewire 260 into posterior tissues of a patient on whom a procedure is being performed.

The right aimer arm 240 shown in FIGS. 10 and 12 includes a second leg 242 (FIG. 10) configured to releasably couple with the base 201 in a common plane with the base 201. In the illustrated embodiment, the knob 211 is located on the base 201 such that when the knob 211 is tightened, sliding movement between the second leg 242 of the right aimer arm 240 along the arc of the second leg 242 is restricted. When the knob 211 is loosened, the second leg 242 is released and allowed to slide relative to the base 201 along the arc of the second leg 242. The second leg 242 of the right aimer arm 240 of the illustrated embodiment is shaped in cooperation with the base 201 such that when the second leg 242 of the right aimer arm 240 is moved along its arc, the distal tip 247 of the right extension 245 remains aligned with an extended central axis of the drill guide 210.

The right aimer arm 240 shown includes the right extension 245 with a second main axis 249 (FIG. 12) that extends from its proximal end 246 to its distal tip 247. In the illustrated embodiment, the proximal end 246 of the right extension 245 is fixed to the second leg 242, a central portion 248 of the right extension 245 diverts to the right of the second main axis 249. The distal tip 247 of the right extension 245 is configured to align with a central axis of the drill guide 210 similar to the alignment shown with the distal tip 237 in FIG. 9. The distal tip 247 of the right extension 245 is configured to receive or “catch” a distal portion of the guidewire 260, which is useful in preventing penetration of the guidewire 260 into posterior tissues of a patient on whom a procedure is being performed.

The femoral fixation assembly of the ligament repair system may include one or more fixation devices, such as but not limited to interference screws, pound-in anchors, and expanding anchors. In some embodiments, an ALB is coupled with a metal interference screw. The metal interference screw may be a nominal 7 mm diameter screw in some embodiments. In some embodiments, a PMB is coupled with a resorbable screw, such as but not limited to a BIOSURE REGENESORB brand screw offered by Smith and Nephew in some embodiments. The resorbable screw may be a nominal 7 mm diameter screw in some embodiments. Other effective combinations of screws or fastener types may be used in other embodiments.

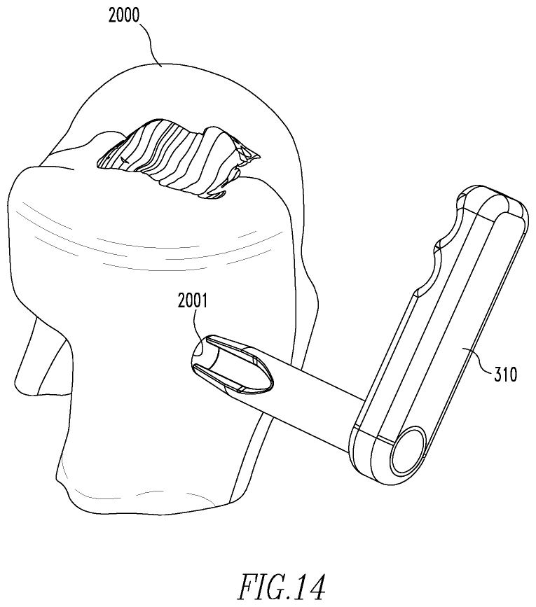

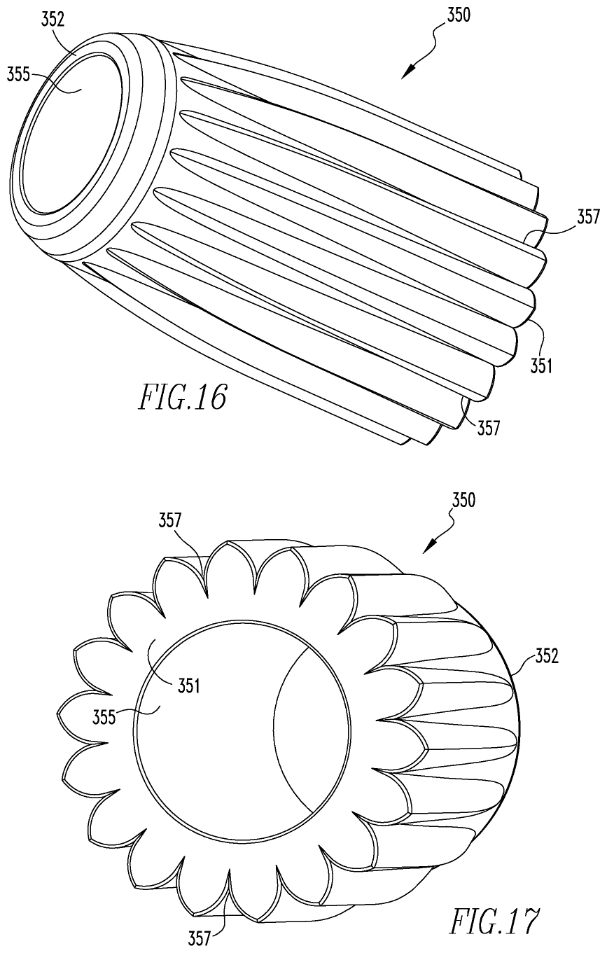

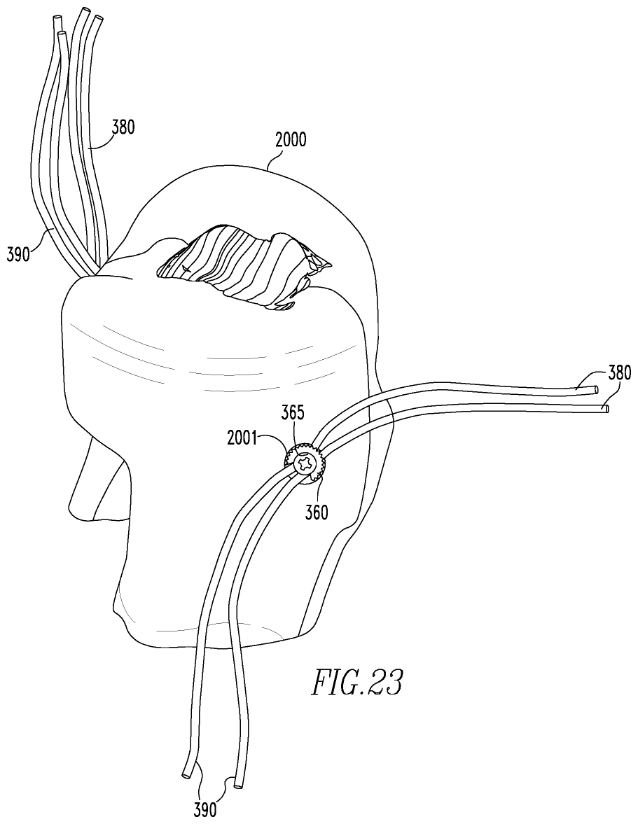

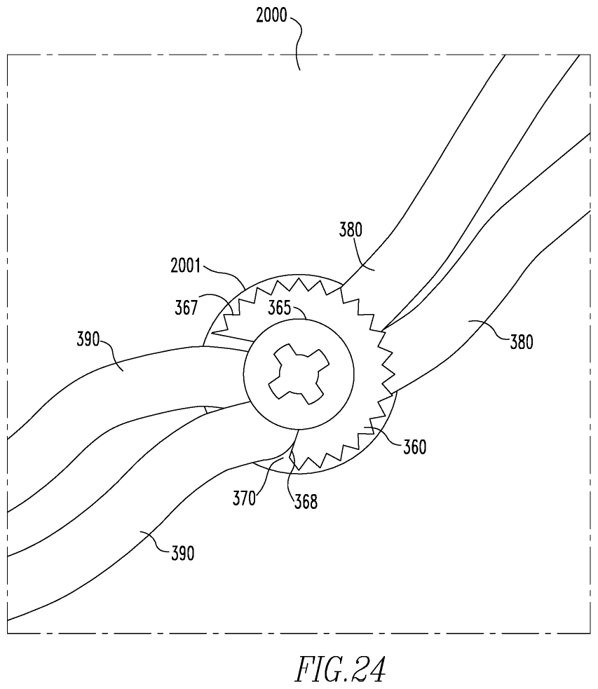

The tibial fixation assembly 300, as shown in multiple embodiments in FIGS. 13-24, includes an exterior anchor 350, 360 and an interior anchor 370. The exterior anchor 350 illustrated in FIGS. 13 and 15-17 has a proximal end 351, a distal end 352, and an interior opening 355. The interior opening 355 of the exterior anchor 350 extends from its proximal end 351 to its distal end 352. The exterior anchor 350 also includes grooves 357 (FIGS. 16 and 17) on its outer surface configured to engage bone, such as the interior of a hole 2001 in the tibia 2000 shown in FIG. 14. The grooves 357 and the exterior anchor 350 generally are configured to couple a graft, ligament, or ligament bundle between an exterior portion of the exterior anchor 350 and a hole in the tibia, such as the hole 2001 in the tibia 2000. A graft, ligament, or ligament bundle may be coupled using the exterior anchor 350 in an essentially similar way to a graft 380 shown being coupled in the hole 2001 in FIGS. 23 and 24. The illustrated graft 380 is a synthetic graft material, such as a woven component, but in other embodiments may be an autograft, allograft, or other structurally sufficient material. The exterior anchor 350 shown is a pound-in anchor, but in other embodiments may be a threaded interference screw, expandable anchor, or other effective anchoring device. The exterior anchor 350 depicted has a continuous annular wall between its proximal end 351 and its distal end 352.

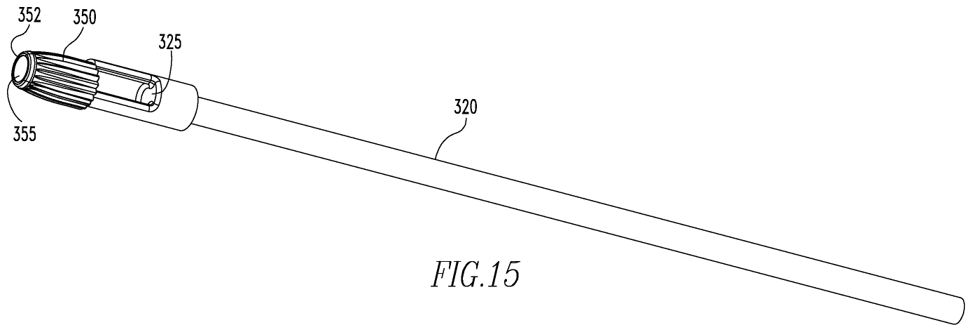

An insertion cannula 310 and an inserter 320 for inserting the exterior anchor 350 are illustrated in FIGS. 13 and 15. The insertion cannula 310 includes a handle 311 and a guide tube 312. The guide tube 312 includes a cutout 313, which provides an opening for a graft, ligament, or ligament bundle to extend through an anchor 350 and an inserter 320 while the insertion cannula 310, inserter 320, and anchor 350 are assembled for insertion. Similarly, the inserter 320 includes a passageway 325 that communicates with the interior opening 355 in the exterior anchor 350 and through which a graft, ligament, or ligament bundle may be passed while the exterior anchor 350 is coupled to the inserter 320. As used to describe coupling between the exterior anchor 350 and the inserter 320, “couple” may include contact between the exterior anchor 350 and the inserter 320, or may include a tensile connection with a stay suture or other component.

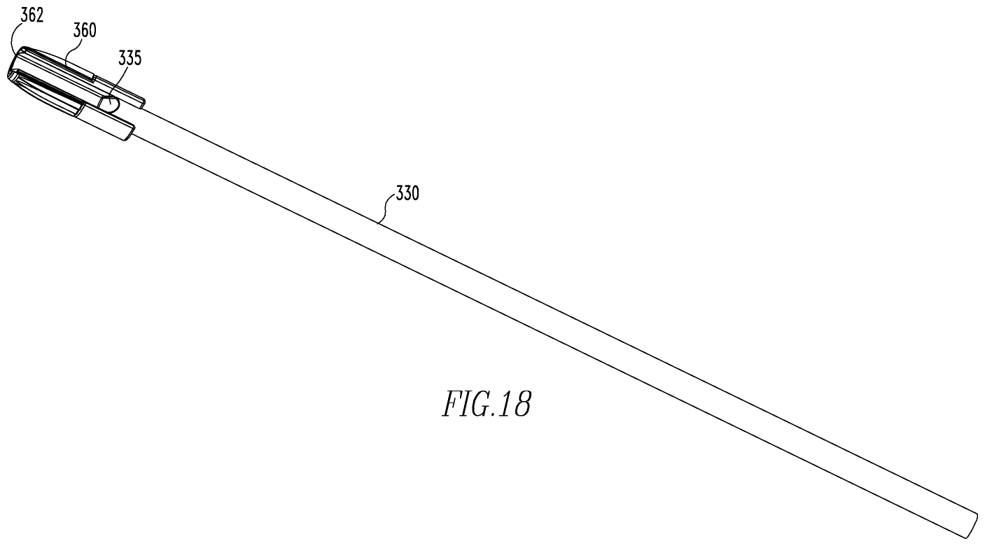

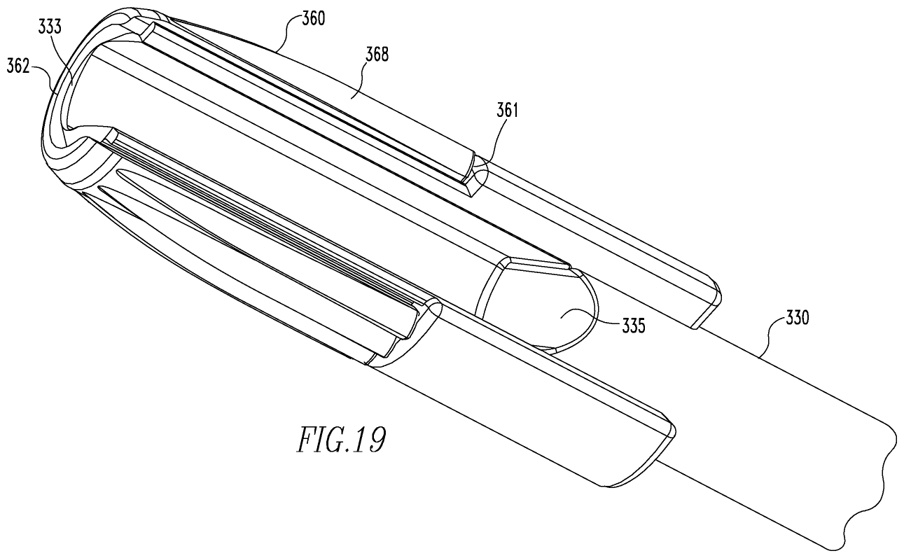

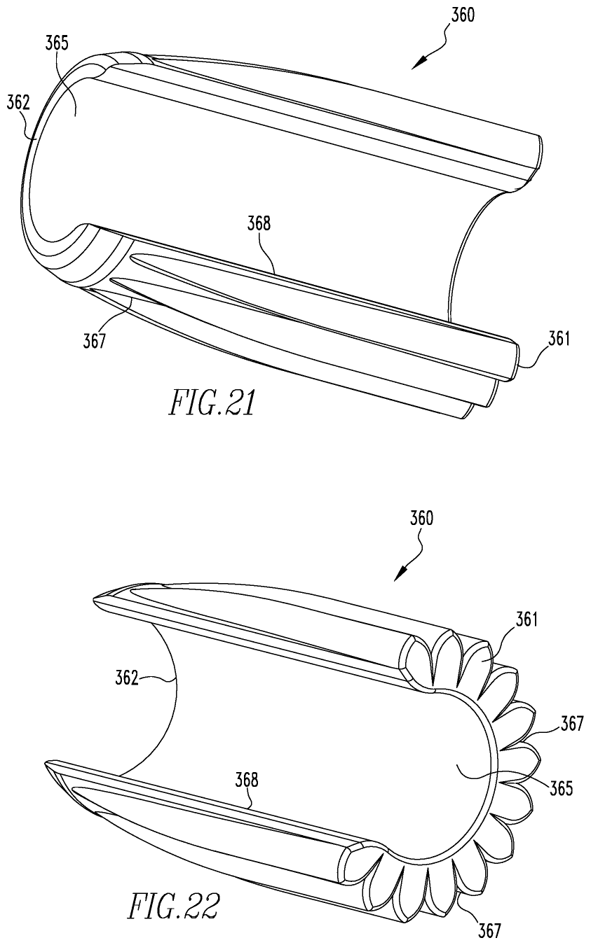

The exterior anchor 360 illustrated in FIGS. 18, 19, and 21-24 has a proximal end 361, a distal end 362, and an interior opening 365. The interior opening 365 of the exterior anchor 360 extends from its proximal end 361 to its distal end 362. The exterior anchor 360 also includes grooves 367 (FIGS. 21 and 22) on its outer surface configured to engage bone, such as the interior of the hole 2001 in the tibia 2000 shown in FIGS. 23 and 24. The grooves 367 and the exterior anchor 360 generally are configured to couple a graft, ligament, or ligament bundle between an exterior portion of the exterior anchor 360 and a hole in the tibia, such as the hole 2001 in the tibia 2000. An example graft 380 coupled in the hole 2001 is illustrated in FIGS. 23 and 24. In the illustrated embodiment, the exterior anchor 360 is a pound-in anchor, but in other embodiments may be a threaded interference screw, expandable anchor, or other effective anchoring device. The exterior anchor 360 depicted has a wall along only a part of a perimeter of the exterior anchor 360 between its proximal end 361 and its distal end 362. The absence of a wall is illustrated in FIGS. 19, 21, and 22 as a slot 368.

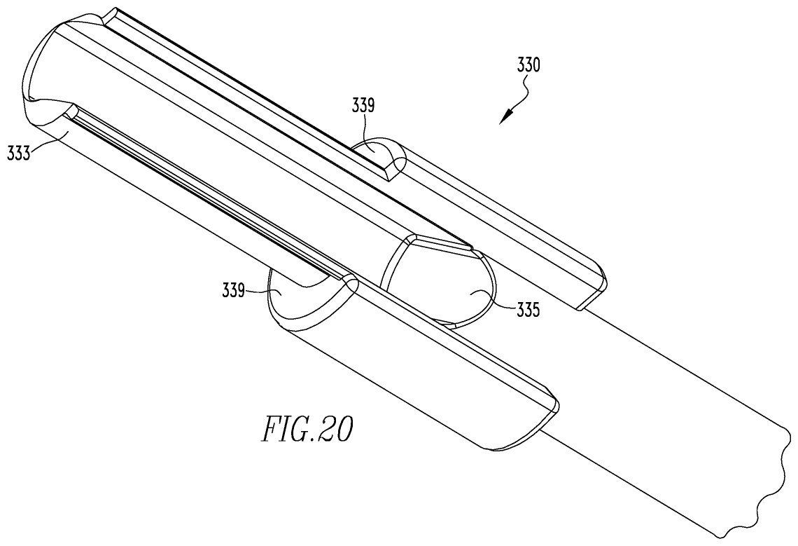

An inserter 330 for inserting the exterior anchor 360 is illustrated in FIGS. 18-20. The inserter 330 includes a passageway 335 that communicates with the interior opening 365 in the exterior anchor 360 and through which a graft, ligament, or ligament bundle may be passed while the exterior anchor 360 is coupled to the inserter 330. As used to describe coupling between the exterior anchor 360 and the inserter 330, “couple” may include contact between the exterior anchor 360 and the inserter 330, or may include tensile connection with a stay suture or other component. As illustrated in most detail in FIGS. 19 and 20, the exterior anchor 360 is coupled with the inserter 330 by the distal end 333 of the inserter 330 being placed within the interior opening 365 of the exterior anchor 360. A shoulder 339 of the inserter 330 provides a surface that may be used to couple with or to push against a proximal end 361 of the exterior anchor 360.

An example internal anchor 370 is depicted in FIG. 24 that fits substantially within the interior opening 365 in the exterior anchor 360 to couple a graft, ligament, or ligament bundle, such as graft 390, relative to the exterior anchor 360. The illustrated graft 390 is a synthetic graft material, such as a woven component, but in other embodiments may be an autograft, allograft, or other structurally sufficient material. The internal anchor 370 shown in FIG. 24 is configured to fix the graft 390 against the tibia 2000 while the graft 390 is in the exterior anchor 360 that has a wall along only a part of a perimeter of the exterior anchor 360 between its proximal end 361 and its distal end 362. In this illustrated embodiment, the interior anchor 370 is actually pressing the graft 390 against the hole 2001 in the tibia 2000 while the interior anchor 370 is in the exterior anchor 360. In other words, in this embodiment, the graft 390 is within the slot 368 (FIGS. 19, 21, and 22) where the interior anchor 370 compresses the graft 390 against the hole 2001 in the tibia 2000.

A method of implanting a PCL replacement graft in a patient's knee is described in context of the devices illustrated in FIGS. 1-24 as follows. Method embodiments may include fixing an ALB graft and a PMB graft to the patient's femur. Fixing the ALB graft and the PMB graft to the patient's femur in some embodiments includes using a femoral aimer assembly, such as the femoral aimer assembly 100 (FIGS. 1-7) to locate a first hole 1001 at an anatomically correct location for the ALB graft. More specifically, as shown in FIG. 6, a distal end of the cannula 120 of the femoral aimer assembly 100 may be positioned adjacent to an anatomically correct location for the ALB graft. In some embodiments, the spur 125 may be used to establish positioning of the cannula 120 relative to the edge 3001 of the cartilage 3000. A guidewire, such as a 2.4 mm k-wire, or other drill may then be advanced through the cannulation 122 (FIG. 5) and into the femur 1000. The hole made by the guidewire or a larger hole reamed over the guidewire or made with the aid of the hole made by the guidewire may be established and used for a fixing location for the ALB graft. A hole or preliminary hole made as shown in FIG. 7 may also serve as a reference for making a second hole in which the PMB graft may be placed. The first hole 1001 may be drilled or drilled and reamed to a final hole diameter of between about 7 mm and 11 mm in some embodiments. The first hole 1001 may be a blind hole between about 20 mm and 25 mm deep.

As shown in FIG. 7, the femoral aimer assembly 100 may be used to create a second hole relative to the first hole 1001 at an anatomically correct location for the PMB by placing a protrusion 127 of the femoral aimer assembly 100 in the first hole 1001 and creating the second hole through the cannulation 122 (FIG. 5) in the femoral aimer assembly 100. The spur 125 may be used to establish positioning of the cannula 120 relative to the edge 3001 of the cartilage 3000 while the protrusion 127 is in the first hole 1001. A guidewire, such as a 2.4 mm k-wire, or other drill may be advanced through the cannulation 122 to create at least part of the second hole. The cannulation 122 may be a predetermined offset distance from the protrusion 127. For example, the offset distance from the cannulation to the protrusion may be enough to leave at least about a 2 mm bone bridge between the first hole 1001 and the second hole after the holes are enlarged to their final sizes. The second hole may be drilled or drilled and reamed to a final hole diameter of between about 7 mm and 11 mm in some embodiments. The second hole may be a blind hole between about 20 mm and 25 mm deep.

Fixing one or both of the ALB graft and the PMB graft to the femur 1000 may include using implants or fixation devices to secure graft connections to the femur 1000. The one or more fixation devices that may be used include but are not limited to interference screws, pound-in anchors, and expanding anchors. In some embodiments, a metal interference screw may be used. The metal interference screw may be a nominal 7 mm diameter in some embodiments. In some embodiments, a resorbable screw, such as but not limited to a BIOSURE REGENESORB brand screw offered by Smith and Nephew may be used. The resorbable screw may be a nominal 7 mm diameter screw in some embodiments. Other effective combinations of screws, fastener types, and fixation devices may be used in other embodiments.

In some embodiments, fixing the ALB graft and the PMB graft to the patient's femur includes pulling one or both of the grafts through an anterolateral portal into the knee joint. Some embodiments may also include passing one or both of the ALB graft and the PMB graft into respective first and second holes in the femur with the aid of one or more guidewires used to create the respective first and second holes in the femur.

The method of implanting a PCL replacement graft in a patient's knee also includes the act of preparing the patient's tibia to receive the ALB graft and the PMB graft. In some embodiments, the act of preparing the patient's tibia to receive the ALB graft and the PMB graft includes drilling a hole in the patient's tibia through a correct anatomical PCL footprint location using an extension on a drill guide that diverts around the patient's tibial eminence and ACL, where the distal tip of the extension provides a drilling location and a target toward which drilling will be accomplished. Some methods may include selecting an extension from a set of extensions that are configured for either right or left diversion around the patient's tibial eminence and ACL. In some embodiments, preparing the patient's tibia includes using the DIRECTOR brand guide system and the ACUFEX brand Tibial Drill Guide, both offered by Smith and Nephew. Preparing the patient's tibia may include locating a distal tip of 237, 247 of a PCL tibial aimer, such as the aimer arms 230, 240, on the center of a native PCL tibial footprint and drilling a guidewire 260, such as a 2.4 mm guidewire, into the tibia at an angle of about 55 degrees. A reamer may be used to widen the hole created by the guidewire. For example and without limitation, a cannulated 12 mm reamer, such as an acorn reamer, may be used to prepare the patient's tibia. With the tibia prepared, the ALB graft and the PMB graft may be positioned through the tibia for fixation. The grafts may be moved through the prepared tibia with, for example and without limitation, a commercially available GORE SMOOTHER or functionally similar device.

The method of implanting a PCL replacement graft in a patient's knee also includes the act of positioning the patient's knee at about 90 degrees of flexion, tensioning the ALB relative to the tibia, and fixing the ALB relative to the tibia by placing an exterior anchor 350, 360 with a proximal end 351, 361, a distal end 352, 362, and an interior opening 355, 365 in a tibial tunnel (hole 2001, FIGS. 14, 23, 24) prepared in the patient's tibia in which the ALB graft 380 (FIGS. 23 and 24) is located. The exterior anchor 350, 360 embodiments are pound-in anchors, but in other embodiments interference screws, expandable anchors, or other effective devices may be placed in a tibial tunnel.

In another act of the method of implanting a PCL replacement graft in a patient's knee, the patient's knee is positioned at about zero degrees of flexion, the PMB is tensioned relative to the tibia, and the PMB (graft 390) is fixed relative to the tibia by placing an interior anchor 370 (FIG. 24) in the interior opening 355, 365 of the exterior anchor 350, 360 in which the PMB is located. In some embodiments, fixing the PMB relative to the tibia by placing the interior anchor 370 in the interior opening 355, 365 of the exterior anchor 350, 360 in which the PMB is located includes fixing the PMB between the exterior anchor 350, 360 and the interior anchor 370. In other embodiments, rather than being fixed between the exterior anchor 350, 360 and the interior anchor 370, the PMB is fixed between a portion of the tibia 2000 exposed through a wall of the exterior anchor 360, such as through the slot 368 (FIGS. 19, 21, and 22), and the interior anchor 370 (FIG. 24). Fixing the PMB relative to the tibia may include using interference screws, pound-in anchors, or expanding anchors. In some embodiments, a metal interference screw may be used. In some embodiments, a resorbable screw, such as but not limited to a BIOSURE REGENESORB brand screw offered by Smith and Nephew may be used.

Various embodiments of an instrument set and implants in whole or in their components individually may be made from any biocompatible material. For example and without limitation, biocompatible materials may include in whole or in part: non-reinforced polymers, reinforced polymers, metals, ceramics, adhesives, reinforced adhesives, and combinations of these materials. Reinforcing of polymers may be accomplished with carbon, metal, or glass or any other effective material. Examples of biocompatible polymer materials include polyamide base resins, polyethylene, low density polyethylene, polymethylmethacrylate (PMMA), polyetheretherketone (PEEK), polyetherketoneketone (PEKK), a polymeric hydroxyethylmethacrylate (PHEMA), and polyurethane, any of which may be reinforced. Example biocompatible metals include stainless steel and other steel alloys, cobalt chrome alloys, zirconium, oxidized zirconium, tantalum, titanium, titanium alloys, titanium-nickel alloys such as Nitinol and other superelastic or shape-memory metal alloys.

Terms such as proximal, distal, against, left, right, and the like have been used relatively herein. However, such terms are not limited to specific coordinate orientations, distances, or sizes, but are used to describe relative positions referencing particular embodiments. Such terms are not generally limiting to the scope of the claims made herein. Any embodiment or feature of any section, portion, or any other component shown or particularly described in relation to various embodiments of similar sections, portions, or components herein may be interchangeably applied to any other similar embodiment or feature shown or described herein.

While embodiments of the disclosure have been illustrated and described in detail in the disclosure, the disclosure is to be considered as illustrative and not restrictive in character. All changes and modifications that come within the spirit of the disclosure are to be considered within the scope of the disclosure.

1. A ligament repair system comprising:
a femoral aimer assembly comprising:
a handle,
a cannula configured to couple with the handle, the cannula having a cannulation with a longitudinal axis through which a guidewire may be inserted into a femur, and
a protrusion at a distal end of the cannula with a center that is offset from a center of the longitudinal axis of the cannulation, wherein when the protrusion is placed in a hole in the femur, the offset distance between the protrusion and the longitudinal axis of the cannulation defines a predetermined distance between the hole and a guidewire that may be inserted through the cannulation;
a tibial aimer assembly comprising:
a base,
a drill guide coupled to the base,
a left aimer arm configured to releasably couple with the base, the left aimer arm comprising:
a first leg configured to releasably couple with the base in a common plane with the base, and
a left extension with a first main axis that extends from its proximal end to its distal tip, wherein the proximal end of the left extension is fixed to the first leg, a central portion of the left extension diverts to the left of the first main axis, and the distal tip of the left extension is configured to align with a central axis of the drill guide, and
a right aimer arm configured to releasably couple with the base, the right aimer arm comprising:
a second leg configured to releasably couple with the base in a common plane with the base, and
a right extension with a second main axis that extends from its proximal end to its distal tip, wherein the proximal end of the right extension is fixed to the second leg, a central portion of the right extension diverts to the right of the second main axis, and the distal tip of the right extension is configured to align with a central axis of the drill guide;
a femoral fixation assembly comprising:
a first fixation device for coupling a first ligament bundle to the femur, and
a second fixation device for coupling a second ligament bundle to the femur; and
a tibial fixation assembly comprising:
an exterior anchor with a proximal end, a distal end, and an interior opening, the exterior anchor configured to couple the first ligament bundle between an exterior portion of the exterior anchor and a hole in a tibia, and
an internal anchor that fits substantially within the interior opening in the exterior anchor to couple the second ligament bundle relative to the exterior anchor.

2. The ligament repair system of claim 1, wherein the longitudinal axis of the cannulation of the femoral aimer assembly is coupled to the handle at an angle of about 45 degrees to a longitudinal axis of the handle.

3. The ligament repair system of claim 1, wherein the longitudinal axis of the cannulation of the femoral aimer assembly is coupled to the handle at an angle of about zero degrees to a longitudinal axis of the handle.

4. The ligament repair system of claim 1, wherein the protrusion of the femoral aimer assembly is substantially the size of the guidewire.

5. The ligament repair system of claim 1, wherein the protrusion of the femoral aimer assembly is substantially the size of a hole reamed to accept a ligament fixation device.

6. The ligament repair system of claim 5, wherein a tool used to create the reamed hole has a diameter between about 7 mm and 11 mm.

7. The ligament repair system of claim 1, wherein the center of the protrusion of the femoral aimer assembly is between about 7 mm and 11 mm from the center of the longitudinal axis of the cannulation.

8. The ligament repair system of claim 1, wherein the femoral aimer assembly is configured and sized such that when a first hole has been made in the femur to accept the protrusion and a second hole has been made in the femur with the protrusion in the first hole, and the first and second holes have been reamed, a bone bridge of about at least 2 mm exists between the first hole and the second hole.

9. The ligament repair system of claim 1, wherein the first leg of the left aimer arm of the tibial aimer releasably couples to the base along an arc such that when the first leg of the left aimer arm is moved along the arc, the distal tip of the left extension remains aligned with an extended central axis of the drill guide.

10. The ligament repair system of claim 1, wherein the second leg of the right aimer arm of the tibial aimer releasably couples to the base along an arc such that when the second leg of the right aimer arm is moved along the arc, the distal tip of the right extension remains aligned with an extended central axis of the drill guide.

11. The ligament repair system of claim 1, wherein the first ligament bundle is allograft.

12. The ligament repair system of claim 1, wherein the first ligament bundle is autograft.

13. The ligament repair system of claim 1, wherein the first ligament bundle is a synthetic graft substitute material.

14. The ligament repair system of claim 1, wherein the second ligament bundle is allograft.

15. The ligament repair system of claim 1, wherein the second ligament bundle is autograft.

16. The ligament repair system of claim 1, wherein the second ligament bundle is a synthetic graft substitute material.

17. The ligament repair system of claim 1, wherein the first and second ligament bundles are joined to each other away from one or more of their respective ends.

18. The ligament repair system of claim 1, wherein the exterior anchor is a pound-in anchor.

19. The ligament repair system of claim 1, wherein the exterior anchor has a wall along only a part of a perimeter of the exterior anchor between its proximal end and its distal end.

20. The ligament repair system of claim 1, wherein the exterior anchor has a continuous annular wall between its proximal end and its distal end.

21. The ligament repair system of claim 1, wherein the exterior anchor has an opening from its proximal end to its distal end.

22. The ligament repair system of claim 1, further comprising an inserter for inserting the exterior anchor, the inserter including a passageway through which the first ligament bundle may be passed while the exterior anchor is coupled to the inserter.

23. The ligament repair system of claim 1, wherein the internal anchor is configured to fix the second ligament against the interior opening in the exterior anchor.

24. The ligament repair system of claim 1, wherein the internal anchor is configured to fix the second ligament bundle against the tibia while the second ligament bundle is in an exterior anchor that has a wall along only a part of a perimeter of the exterior anchor between its proximal end and its distal end.

25. A femoral aimer assembly comprising:
a handle;
a cannula configured to couple with the handle, the cannula having a cannulation with a longitudinal axis through which a guidewire may be inserted into a femur; and
a protrusion at a distal end of the cannula with a center that is offset from a center of the longitudinal axis of the cannulation, wherein when the protrusion is placed in a hole in the femur, the offset distance between the protrusion and the longitudinal axis of the cannulation defines a predetermined distance between the hole and a guidewire that may be inserted through the cannulation.

26. The femoral aimer assembly of claim 25, wherein the longitudinal axis of the cannulation of the femoral aimer assembly is coupled to the handle at an angle of about 45 degrees to a longitudinal axis of the handle.

27. The femoral aimer assembly of claim 25, wherein the longitudinal axis of the cannulation of the femoral aimer assembly is coupled to the handle at an angle of about zero degrees to a longitudinal axis of the handle.

28. The femoral aimer assembly of claim 25, wherein the protrusion is substantially the size of the guidewire.

29. The femoral aimer assembly of claim 25, wherein the protrusion is substantially the size of a hole reamed to accept a ligament fixation device.

30. The femoral aimer assembly of claim 29, wherein a tool used to create the reamed hole has a diameter between about 7 mm and 11 mm.

31. The femoral aimer assembly of claim 25, wherein the center of the protrusion is between about 7 mm and 11 mm from the center of the longitudinal axis of the cannulation.

32. The femoral aimer assembly of claim 25, wherein the femoral aimer assembly is configured and sized such that when a first hole has been made in the femur to accept the protrusion and a second hole has been made in the femur with the protrusion in the first hole, and the first and second holes have been reamed, a bone bridge of about at least 2 mm exists between the first hole and the second hole.

33. The femoral aimer assembly of claim 25, further comprising a first spur near the distal end of the cannula that extends away from the longitudinal axis of the cannula and is substantially perpendicular to a length of the protrusion.

34. The femoral aimer assembly of claim 33, further comprising a second spur near the distal end of the cannula that extends away from the longitudinal axis of the cannula opposite from the first spur and is substantially perpendicular to a length of the protrusion.

35. A tibial aimer assembly comprising:
a base;
a drill guide coupled to the base;
a left aimer arm configured to releasably couple with the base, the left aimer arm comprising:
a first leg configured to releasably couple with the base in a common plane with the base, and
a left extension with a first main axis that extends from its proximal end to its distal tip, wherein the proximal end of the left extension is fixed to the first leg, a central portion of the left extension diverts to the left of the first main axis, and the distal tip of the left extension is configured to align with a central axis of the drill guide; and
a right aimer arm configured to releasably couple with the base, the right aimer arm comprising:
a second leg configured to releasably couple with the base in a common plane with the base, and
a right extension with a second main axis that extends from its proximal end to its distal tip, wherein the proximal end of the right extension is fixed to the second leg, a central portion of the right extension diverts to the right of the second main axis, and the distal tip of the right extension is configured to align with a central axis of the drill guide.

36. The tibial aimer assembly of claim 35, wherein the first leg of the left aimer arm of the tibial aimer releasably couples to the base along an arc such that when the first leg of the left aimer arm is moved along the arc, the distal tip of the left extension remains aligned with an extended central axis of the drill guide.

37. The tibial aimer assembly of claim 35, wherein the second leg of the right aimer arm of the tibial aimer releasably couples to the base along an arc such that when the second leg of the right aimer arm is moved along the arc, the distal tip of the right extension remains aligned with an extended central axis of the drill guide.

38. A tibial fixation assembly comprising:
an exterior anchor with a proximal end, a distal end, and an interior opening, the exterior anchor configured to couple a first ligament bundle between an exterior portion of the exterior anchor and a hole in a tibia, and
an internal anchor that fits substantially within the interior opening in the exterior anchor to couple a second ligament bundle relative to the exterior anchor.

39. The tibial fixation assembly of claim 38, wherein the exterior anchor is a pound-in anchor.

40. The tibial fixation assembly of claim 38, wherein the exterior anchor has a wall along only a part of a perimeter of the exterior anchor between its proximal end and its distal end.

41. The tibial fixation assembly of claim 38, wherein the exterior anchor has a continuous annular wall between its proximal end and its distal end.

42. The tibial fixation assembly of claim 38, wherein the exterior anchor has an opening from its proximal end to its distal end.

43. The tibial fixation assembly of claim 38, further comprising an inserter for inserting the exterior anchor, the inserter including a passageway through which the first ligament bundle may be passed while the exterior anchor is coupled to the inserter.

44. The tibial fixation assembly of claim 38, wherein the internal anchor is configured to fix the second ligament against the interior opening in the exterior anchor.

45. The tibial fixation assembly of claim 38, wherein the internal anchor is configured to fix the second ligament bundle against the tibia while the second ligament bundle is in an exterior anchor that has a wall along only a part of a perimeter of the exterior anchor between its proximal end and its distal end.

46. A method of implanting a posterior cruciate ligament (PCL) replacement graft in a patient's knee comprising:
fixing an anterolateral bundle (ALB) graft and a posteromedial bundle (PMB) graft to the patient's femur;
preparing the patient's tibia to receive the ALB graft and the PMB graft;
positioning the ALB graft and the PMB graft through the tibia for fixation;
positioning the patient's knee at about 90 degrees of flexion, tensioning the ALB relative to the tibia, and fixing the ALB relative to the tibia by placing an exterior anchor with a proximal end, a distal end, and an interior opening in a tibial tunnel prepared in the patient's tibia in which the ALB is located; and
positioning the patient's knee at about zero degrees of flexion, tensioning the PMB relative to the tibia, and fixing the PMB relative to the tibia by placing an interior anchor in the interior opening of the exterior anchor in which the PMB is located.

47. The method of claim 46, wherein fixing the ALB graft and the PMB graft to the patient's femur includes placing the ALB and the PMB in blind holes.

48. The method of claim 46, wherein fixing the ALB graft and the PMB graft to the patient's femur includes using a femoral aimer assembly to locate a first hole at an anatomically correct location for an ALB.

49. The method of claim 48, wherein fixing the ALB graft and the PMB graft to the patient's femur includes using the femoral aimer assembly to create a second hole relative to the first hole at an anatomically correct location for a PMB by placing a protrusion of the femoral aimer assembly in the first hole and creating the second hole through a cannulation in the femoral aimer assembly, wherein the cannulation is a predetermined offset distance from the protrusion.

50. The method of claim 49, wherein using the femoral aimer assembly to locate the first hole at an anatomically correct location for an ALB includes using a spur near the distal end of the femoral aimer assembly to estimate the location of the femoral aimer assembly from an edge of a cartilage to bone interface, and using the femoral aimer assembly to locate the hole at an anatomically correct location for a PMB includes using the spur near the distal end of the femoral aimer assembly to estimate the location of the femoral aimer assembly from an edge of a cartilage to bone interface.

51. The method of claim 46, wherein fixing the ALB graft and the PMB graft to the patient's femur includes pulling one or both of the grafts through an anterolateral portal and passing the one or more grafts into respective holes in the femur with the aid of one or more guidewires used to create the respective holes in the femur.

52. The method of claim 46, wherein preparing the patient's tibia to receive the ALB graft and the PMB graft includes drilling a hole in the patient's tibia through a correct anatomical PCL footprint location using an extension on a drill guide that diverts around the patient's tibial eminence and ACL.

53. The method of claim 46, wherein fixing the ALB relative to the tibia by placing an exterior anchor may include implanting a pound-in anchor.

54. The method of claim 46, wherein fixing the ALB relative to the tibia by placing an exterior anchor includes implanting an anchor with a wall along only a part of a perimeter of the exterior anchor between its proximal end and its distal end.

55. The method of claim 46, wherein fixing the ALB relative to the tibia by placing an exterior anchor includes implanting an anchor with a continuous annular wall between its proximal end and its distal end.

56. The method of claim 46, wherein fixing the PMB relative to the tibia by placing the interior anchor in the interior opening of the exterior anchor in which the PMB is located includes fixing the PMB between the exterior anchor and the interior anchor.

57. The method of claim 46, wherein fixing the PMB relative to the tibia by placing the interior anchor in the interior opening of the exterior anchor in which the PMB is located includes fixing the PMB between a portion of the tibia exposed through a wall of the exterior anchor and the interior anchor.
